(19) United States
(12) Patent Application Publication
(10) Pub. No.: US 2019/0298432 A1
(43) Pub. Date: Oct. 3, 2019

(54) ENERGY TREATMENT INSTRUMENT

(71) Applicant: OLYMPUS CORPORATION, Tokyo (JP)

(72) Inventor: Tsunetaka AKAGANE, Hachioji-shi (JP)

(73) Assignee: OLYMPUS CORPORATION, Tokyo (JP)

(21) Appl. No.: 16/414,923

(22) Filed: May 17, 2019

Related U.S. Application Data
(63) Continuation of application No. PCT/JP2016/084323, filed on Nov. 18, 2016.

(51) Int. Cl. A61B 18/08; A61B 18/14; A61B 46/23

(52) U.S. Cl. CPC A61B 18/08; A61B 2018/1455; A61B 46/23; A61B 18/14

(57) ABSTRACT

An energy treatment instrument including a first grasping piece including a base plate located on an inner side of the first grasping piece, and a second grasping piece configured to open and close with respect to the first grasping piece. A first opposing face is located on an outer surface of the base plate and on the inner side of the first grasping piece and facing the second grasping piece. A second opposing face is located on an outer surface of the second grasping piece and facing the first grasping piece. A heat generator is embedded inside the first grasping piece and includes a metal component, where the heat generator generates heat based on a current flowing through the metal component that is to the first opposing face.

### CROSS-REFERENCE TO RELATED APPLICATIONS

This is a Continuation Application of PCT Application No. PCT/JP2016/084323, filed Nov. 18, 2016, the entire contents of which are incorporated herein by reference.

### BACKGROUND

### 1. Field of the Invention

The exemplary embodiments relate to an energy treatment instrument for treating a treatment target using heat.

### 2. Description of the Related Art

The related art discloses an energy treatment instrument for treating a treatment target such as a biological tissue to be grasped between a pair of grasping pieces. In this energy treatment instrument, heat generated in a heat generating element and high-frequency current are applied to a treatment target grasped between a pair of grasping pieces. The treatment target is coagulated and/or incised by the heat and the high-frequency current.

### BRIEF SUMMARY

According to one aspect of the exemplary embodiments, an energy treatment instrument includes a first grasping piece, a second grasping piece configured to open and close with respect to the first grasping piece, a first opposing face that faces the second grasping piece on an outer surface of the first grasping piece, a second opposing face that faces the first grasping piece on an outer surface of the second grasping piece, a heat generator that contains at least a metal component and that is configured to generate heat by a current flowing, and a base plate that is provided in the first grasping piece, the base plate being formed of ceramics having electrical insulation properties, and the base plate being configured to transfer the heat generated by the heat generator, the heat generator being fixed to the base plate in close contact.

Advantages of the exemplary embodiments will be set forth in the description which follows, and in part will be obvious from the description. The advantages of the exemplary embodiments may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate exemplary embodiments, and together with the general description given above and the detailed description of the embodiments given below, serve to explain the principles of the exemplary embodiments.

FIG. 1 is a schematic view showing a treatment system in which an energy treatment instrument according to a first embodiment is used.

FIG. 2 is a view schematically showing a cross section substantially perpendicular to a longitudinal axis of an end effector according to the first embodiment.

FIG. 3 is a view schematically showing a state in which a living tissue is grasped by the end effector according to the first embodiment.

FIG. 4 is a view schematically showing a cross section substantially perpendicular to a longitudinal axis of an end effector according to a first modification of the first embodiment.

FIG. 5 is a view schematically showing a cross section substantially perpendicular to a longitudinal axis of an end effector according to a second modification of the first embodiment.

FIG. 6 is a view schematically showing a cross section substantially perpendicular to a longitudinal axis of an end effector according to a third modification of the first embodiment.

FIG. 7 is a view schematically showing a cross section substantially perpendicular to a longitudinal axis of an end effector according to a fourth modification of the first embodiment.

### DETAILED DESCRIPTION

### First Embodiment

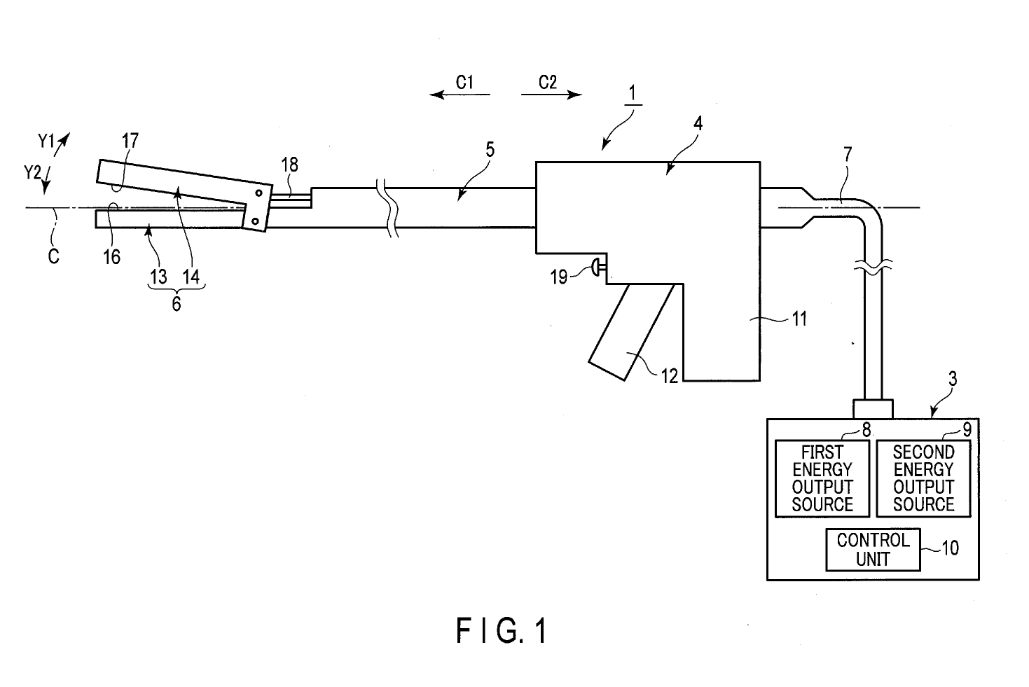

A first embodiment will be described with reference to FIGS. 1 to 3. FIG. 1 is a diagram showing a treatment system in which an energy treatment instrument 1 according to this embodiment is used. As shown in FIG. 1, the energy treatment instrument 1 has a longitudinal axis C. Here, in the energy treatment instrument 1, the direction along the longitudinal axis C is defined as the longitudinal direction. Also, one side in the longitudinal direction is referred to as a distal side (arrow C1 side), and the opposite side to the distal side is referred to as a proximal side (arrow C2 side). In the present embodiment, the energy treatment instrument 1 is a thermal treatment instrument for treating a grasped treatment target by using heat. The energy treatment instrument 1 is also a bipolar high frequency treatment tool that includes two treatment electrodes, and treats the grasped treatment target using a high-frequency current (high frequency energy) flowing between these electrodes.

The energy treatment instrument 1 comprises a housing 4 that is holdable, a shaft 5 connected to the distal portion of the housing 4, and an end effector 6 provided at a distal end portion of the shaft 5. One end of a cable 7 is connected to the housing 4. The other end of the cable 7 is separably connected to a power supply unit 3. The power supply unit 3 includes a first energy output source 8, a second energy output source 9, and a control unit 10. The first energy output source 8 includes a conversion circuit or the like configured to convert electric power from a battery power source or an outlet power source into electric energy (DC power or AC power) to be supplied to a heat generating element (heat source) to be described later, and outputs the converted electric energy. The second energy output source 9 includes a conversion circuit or the like configured to convert electric power from a battery power source or an outlet power source into electric energy (high-frequency power) to be supplied to an electrode to be described later, and outputs the converted electric energy. The control unit 10 includes an integrated circuit or processor including a CPU (Central Processing Unit), an ASIC (Application Specific Integrated Circuit), an FPGA (Field Programmable Gate Array), or the like, and also includes a storage medium.

An operation button 19 as an energy operation input unit is attached to the housing 4. By depressing the operation button 19, an operation (signal) is input to the power supply unit 3 for outputting electric energy from the first energy output source 8 and/or the second energy output source 9 to the energy treatment instrument 1. Instead of or in addition to the operation button 19, a foot switch or the like separate from the energy treatment instrument 1 may be provided as an energy operation input unit.

In the housing 4, a grip (fixed handle) 11 is provided, and a handle (movable handle) 12 is rotatably attached. As the handle 12 rotates relative to the housing 4, the handle 12 opens or closes with respect to the grip 11. In the present embodiment, the handle 12 is located in a distal side of the grip 11 and moves substantially parallel to the longitudinal axis C in an opening or closing operation with respect to the grip 11. However, the handle 12 is not limited to this embodiment. For example, in one embodiment, the handle 12 may be located in a proximal side of the grip 11. In another embodiment, the handle 12 may be located on the side opposite the grip 11 with respect to the longitudinal axis C, and the direction of movement in the opening or closing movement with respect to the grip 11 may intersect (perpendicular to) the longitudinal axis C.

The shaft 5 extends along the longitudinal axis C. Further, the end effector 6 comprises a first grasping piece 13 and a second grasping piece 14 (jaw) which opens and closes relative to the first grasping piece 13. The outer surface of the first grasping piece 13 comprises a first opposing face 16 which faces the second grasping piece 14. The outer surface of the second grasping piece 14 has a second opposing face 17 which faces the first opposing face 16 of the first grasping piece 13. The handle 12 and the second grasping piece 14 are connected via a movable member 18 that extends along the longitudinal axis C inside the shaft 5. By opening or closing the handle 12, which is an opening/closing operation input portion, with respect to the grip 11, the movable member 18 moves along the longitudinal axis C with respect to the shaft 5 and the housing 4, so that the pair of grasping pieces 13 and 14 open or close. By closing the grasping pieces 13 and 14, a living tissue such as a blood vessel is grasped as a treatment target between the first grasping piece 13 and the second grasping piece 14. In the state where the grasping pieces 13 and 14 are closed, the grasping pieces 13 and 14 are extended along the longitudinal direction. In the present embodiment, the first grasping piece 13 is fixed to the shaft 5 and the second grasping piece 14 is pivotally attached to the distal end portion of the shaft 5.

The opening and closing directions of the end effector 6 intersect (perpendicular to) the longitudinal axis C. Of the opening and closing directions of the end effector 6, the direction in which the second grasping piece 14 opens with respect to the first grasping piece 13 is defined as an opening direction of the second grasping piece 14 (the direction of the arrow Yl in FIG. 1), and the direction in which the second grasping piece 14 closes with respect to the first grasping piece 13 is defined as a closing direction of the second grasping piece 14 (the direction of the arrow Y2 in FIG. 1). Further, the direction crossing the longitudinal axis C and intersecting with the opening and closing directions of the second grasping piece 14 is defined as a width direction of the end effector 6 (the first grasping piece 13 and the second grasping piece 14).

The configuration can be such that the first grasping piece 13 and the second grasping piece 14 are provided at the distal end portion of the shaft 5 and the first grasping piece 13 and the second grasping piece 14 can be opened and closed. For example, in one embodiment, the first grasping piece 13 is integrally formed with the shaft 5. The second grasping piece 14 is then pivotally attached to the distal end portion of the shaft 5. In another embodiment, both the first grasping piece 13 and the second grasping piece 14 are pivotally attached to the distal end portion of the shaft 5.

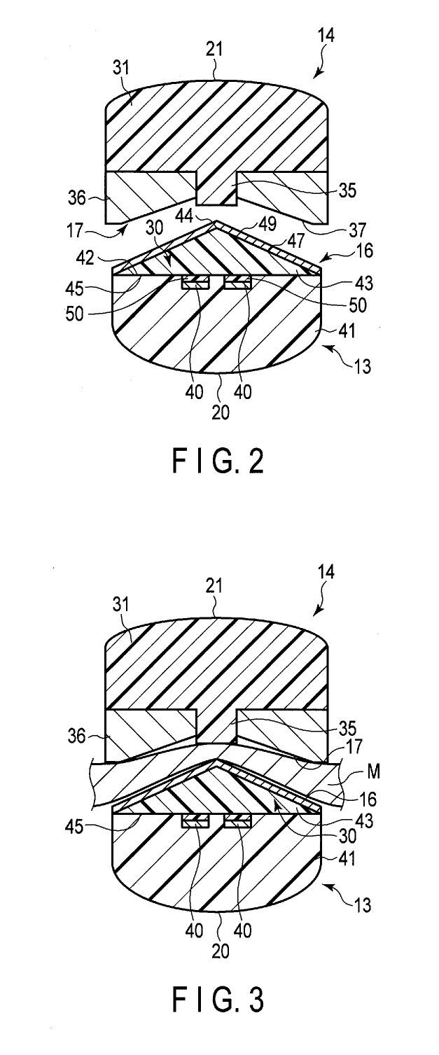

FIG. 2 is a diagram showing the first grasping piece 13 and the second grasping piece 14. FIG. 2 shows a cross section substantially perpendicular to the longitudinal axis C. As shown in FIG. 2, the first grasping piece 13 has a base (base material: support member: structure maintaining member) 41. The base 41 is formed of a material having low thermal conductivity and low electric conductivity (that is, high electric resistance). Further, the base 41 is preferably made of a material having electrical insulation properties. The base 41 is made of, for example, a material containing a heat-resistant resin. Examples of the heat-resistant resin forming the base 41 include engineering plastics, super engineering plastics and the like, and examples thereof include PEEK (polyetheretherketone), LCP (liquid crystal polymer), PFA (perfluoroalkoxyalkane), and the like. In the present embodiment, the base 41 is electrically insulative. The base 41 is extended along the extension direction of the first grasping piece 13. The base 41 has a support surface 42 facing the second grasping piece 14 side and a back surface 20 facing in direction opposite to the support surface 42. The back surface 20 is a surface facing away from the first opposing face 16 on the outer surface of the first grasping piece 13. The back surface 20 is exposed to the outside in the first grasping piece 13.

On a side of the base 41 facing the second grasping piece 14, a heat generating portion (heat generating unit) 30 is fixed. The heat generating portion 30 includes a substrate member (heat transfer member/base plate) 43. The substrate member (heat transfer member/base plate) 43 is attached to the support surface 42 of the base 41. The substrate member 43 is formed of a member having a thermal conductivity higher than that of the base 41. That is, the base 41 has a lower thermal conductivity than the substrate member 43. As the substrate member 43, for example, ceramics such as aluminum nitride is used. Furthermore, the substrate member 43 preferably has electrical insulation properties.

The substrate member 43 has a substrate facing surface 47 that faces the second grasping piece 14 side. In the present embodiment, the first opposing face 16 is formed by the substrate facing surface 47. The substrate facing surface 47 is inclined in the width direction of the first grasping piece 13 so that the substrate facing surface 47 incline toward the second grasping piece 14 side from the outside toward the center in the width direction. Therefore, a protruding portion 44 protruding toward the second grasping piece 14 side is formed in the central portion in the width direction of the first opposing face 16.

The substrate member 43 has a bottom surface 45 that faces the side opposite to the substrate facing surface 47. The bottom surface 45 is a surface that faces away from the first opposing face 16. The bottom surface 45 is in contact with the support surface 42 of the base 41 from the side of the second grasping piece 14.

Further, the heat generating portion 30 includes a heat generating element (heat source) 40. The heat generating element 40 is provided between the support surface 42 of the base 41 and the bottom surface 45 of the substrate member 43. The heat generating element (heat generator) 40 is fixed to the bottom surface 45 of the substrate member 43 so as to be in close contact from the back surface 20 side. As the heat generating element 40, for example, a metal coating such as gold, silver, copper, platinum or the like is used. Thus, the heat generating element 40 contains a metal component. As the metal coating, platinum is particularly preferable. The heat generating element 40 is formed on the bottom surface 45 by, for example, sputtering. Further, the metal wire formed of the metal described above may be disposed on the bottom surface 45 of the substrate member 43 as the heat generating element 40.

As described above, since the heat generating element 40 is formed of a metal, the heat generating element 40 is formed of a material having a high electric conductivity (that is, having a low electric resistance) as compared with the base 41 and the substrate member 43. Therefore, each of the base 41 and the substrate member 43 has a lower electric conductivity (higher electric resistance) than the heat generating element 40. Note that the heat generating element 40 may be in close contact with the support surface 42 of the base 41 or an appropriate space may be provided between the heat generating element 40 and the support surface 42 of the base 41.

The heat generating element 40 is electrically connected to the first energy output source 8 of the power supply unit 3 via an electrical path (not shown) extending through the inside of the first grasping piece 13, the inside of the shaft 5, the inside of the housing 4 and the inside of the cable 7. By supplying electric energy (DC power or AC power) from the power supply unit 3 to the heat generating element (heat source) 40 via this electrical path, heat is generated in the heat generating element 40. The heat generated in the heat generating element 40 is transferred to the substrate member 43 via the bottom surface 45. In other words, in the heat generating element 40, heat is generated as a current flows, and the generated heat is directly transferred from the heat generating element 40 to the substrate member 43. The heat transferred to the substrate member 43 is transferred to the substrate facing surface 47 forming the first opposing face 16 via the interior of the substrate member 43. The base 41 has a lower thermal conductivity than the heat generating element 40 and the substrate member 43. For this reason, the heat generated by the heat generating element 40 is not easily transferred to the base 41.

The bottom surface 45 of the substrate member 43 is coated with an insulating coating 50. The insulating coating 50 is a thin film having electrical insulation properties. As the insulating coating 50, for example, a ceramic coating or a heat-resistant resin such as PEEK, LCP, fluorine resin, parylene or the like is used. The insulating coating 50 is provided between the substrate member 43 and the heat generating element 40. Therefore, electrical insulation properties between the heat generating element 40 and the substrate member 43 are further improved. Therefore, the current flowing through the heat generating element 40 is prevented from flowing into the substrate member 43 even at a higher voltage (electric energy). If the insulating coating 50 is not provided, it is preferable that the substrate member 43 has electrical insulation properties. Further, in the present embodiment, the base 41 has electrical insulation properties. Therefore, the base 41 and the heat generating element 40 are electrically insulated from each other even if the insulating coating 50 is not provided. Thus, the current flowing through the heat generating element 40 is prevented from flowing into the base 41.

As long as one or more heat generating elements 40 are arranged on the bottom surface 45 of the substrate member 43, the number of heat generating elements 40, the extension pattern of heat generating elements 40, and the like are not limited.

The substrate facing surface 47 of the substrate member 43 forming the first opposing face 16 is coated with a conductive coating 49 along the longitudinal direction. The conductive film 49 is provided on the outer surface of the substrate member 43. The conductive coating 49 is a thin film formed of a coating material having water repellency and electrical conductivity. The conductive coating 49 is formed of a material having a thermal conductivity higher than that of the base 41. As the conductive coating 49, for example, metal plating, a mixture material of a fluororesin and a metal powder (Ag, Ni, etc.), or the like is used. In the present embodiment, the conductive film 49 is in close contact with all of the substrate facing surface 47 of the substrate member 43 forming the first opposing face 16 from the second grasping piece 14 side.

The conductive coating 49 is electrically connected to the second energy output source 9 of the power supply unit 3 via an electrical path (not shown) extending through the inside of the first grasping piece 13, the inside of the shaft 5, the inside of the housing 4 and the inside of the cable 7. The conductive coating 49 functions as a (first) electrode by supply of electric energy (high frequency power) from the second energy output source 9. Here, the substrate member 43 and the base 41 have electrical insulation properties. Therefore, the electric energy from the second energy output source 9 is not supplied (transferred) to the substrate member 43 and the base 41.

The second grasping piece 14 includes a support member 31. The support member 31 extends in the longitudinal direction. The support member 31 has electrical insulation properties. As the support member 31, for example, a heat-resistant resin such as PTFE (polytetrafluoroethylene) is used. On the outer surface of the second grasping piece 14, the support member 31 forms a back surface 21, which faces away from the second opposing face 17. The back surface 21 is exposed to the outside in the second grasping piece 14.

A conductive member (an electrical conductive member) 36 is fixed to a side of the first grasping piece 13 of the support member 31. The conductive member 36 is fixed to the support member 31 from the side of the first grasping piece 13. The conductive member 36 extends along the extending direction of the second grasping piece 14 from the proximal end portion to the distal end portion of the second grasping piece 14. The conductive member 36 is formed of a material having electrical conductivity such as metal. The conductive member 36 has an electrode surface 37 facing the first grasping piece 13 side. The electrode surface 37 forms a part of the outer surface of the second grasping piece 14. In addition, the electrode surface 37 forms a part of the second opposing face 17.

The conductive member 36 is electrically connected to the second energy output source 9 of the power supply unit 3 via an electrical path (not shown) extending through the inside of the second grasping piece 14, the inside of the shaft 5, the inside of the housing 4 and the inside of the cable 7. The conductive member 36 functions as a (second) electrode different from the first electrode provided in the first grasping piece 13 by supply of electric energy (high frequency power) from the second energy output source 9. Here, the support member 31 has electrical insulation properties. Therefore, the electric energy from the second energy output source 9 is not supplied (transferred) to the support member 31.

The support member 31 has a protruding portion 35 protruding toward the first grasping piece 13 side through the gap in the conductive member 36. The protruding portion 35 is exposed to the outside through the electrode surface 37 of the conductive member 36. The second opposing face 17 is formed by the electrode surface 37 of the conductive member 36 and the protruding portion 35 of the base 41. The protruding portion 35 is provided in the central portion of the second opposing face 17 in the width direction.

The electrode surface 37 is positioned on both outer sides of the protruding portion 35 in the width direction. The electrode surface 37 is formed so as to incline toward the first grasping piece 13 side from the center toward the outside in the width direction. The electrode surface 37 is a slope (inclined surface) inclined with respect to the width direction.

When the first grasping piece 13 and the second grasping piece 14 are closed in a state in which a treatment target is not placed between the first grasping piece 13 and the second grasping piece 14, the protruding portion 44 of the first opposing face 16 is brought into contact with the protruding portion 35 of the second opposing face 17. At this time, the first opposing face 16 and the electrode surface 37 of the second opposing face 17 are not in contact with each other. Therefore, the conductive coating 49 provided on the substrate facing surface 47 is not brought into contact with the conductive member 36. Therefore, a short circuit due to contact between the conductive film 49 which is the first electrode and the conductive member 36 which is the second electrode is prevented.

The inclination angle of the second opposing face 17 with respect to the width direction is set to be smaller than the inclination angle of the first opposing face 16 with respect to the width direction. That is, the inclination angles are set such that the incised treatment target between the first opposing face 16 and the second opposing face 17 can easily move outward from the central portion in the width direction.

Next, effects and advantages of the energy treatment instrument 1 of the present embodiment will be described with reference to FIGS. 1 to 3. When performing treatment using the energy treatment instrument 1, the surgeon holds the housing 4 of the energy treatment instrument 1 and inserts the end effector 6 into a body cavity such as an abdominal cavity or the like. Then, the surgeon places a treatment target such as a blood vessel between the grasping pieces 13 and 14, and closes the handle 12 relative to the grip 11 to close the grasping pieces 13 and 14. As a result, living tissues such as blood vessels are grasped between the grasping pieces 13 and 14.

FIG. 3 is a view showing a state in which a living tissue M is grasped between the grasping pieces 13 and 14. In this state, the operation input is performed at the energy operation input unit (operation button 19), thereby supplying electric energy from the first energy output source 8 to the heat generating element 40. By supplying electric energy to the heat generating element 40, heat is generated in the heat generating element 40. The heat generated in the heat generating element 40 is transferred to the substrate member 43 via the bottom surface 45. Then, the transferred heat is applied to the living tissue M via the first opposing face 16 formed by the substrate facing surface 47 of the substrate member 43. As a result, the heat is applied to the living tissue M grasped between the first opposing face 16 and the second opposing face 17. As the heat is applied, the grasped living tissue M is incised and at the same time coagulated.

In addition, by performing the operation input at the energy operation input unit (the operation button 19), electric energy (high-frequency power) is supplied from the second energy output source 9 to each of the conductive film 49 which is the first electrode and the conductive member 36 which is the second electrode. By supplying electric energy to each of the conductive coating 49 and the conductive member 36, a high-frequency current flows between the first opposing face 16 and the electrode surface 37 of the second opposing face 17 through the grasped living tissue M. As a result, the high-frequency current is applied to the living tissue M grasped between the first opposing face 16 and the second opposing face 17. That is, the high-frequency energy is supplied to the part between the first opposing face 16 and the second opposing face 17. By applying the high-frequency current, coagulation of the grasped living tissue M is promoted. As described above, the first opposing face 16 and the second opposing face 17 serve as treatment surfaces for treating the grasped treatment target.

Here, the heat generated by the heat generating element 40 is applied to the living tissue M grasped by the first opposing face 16 via the substrate facing surface 47 of the substrate member 43. Therefore, the substrate member 43 forms a portion that applies heat to the treatment target on the first opposing face 16. In the present embodiment, the heat generating element 40 is directly attached to the substrate member 43 without any other member interposed therebetween. Therefore, the heat from the heat generating element 40 is directly transferred to the member forming the portion that applies heat to the living tissue M in the first opposing face 16. For this reason, the heat path formed between the heat generating element 40 and the substrate member 43 is shorter in comparison with the case where another member is present between the heat generating element 40 and the substrate member 43. Furthermore, the heat from the heat generating element 40 is transferred to the substrate facing surface 47 via only the substrate member 43. Therefore, in comparison with the case where another member is present between the heat generating element 40 and the substrate member 43 in the heat transfer path, the loss of thermal energy at the boundary portion between the members is reduced. As a result, the heat can be efficiently transferred from the heat generating element 40 to the portion that applies heat in the first opposing face 16 and to the treatment target.

In the case where the portion that applies heat to the living tissue grasped by the first opposing face 16 is formed by another member different from the substrate member 43, warpage or breakage may occur at the boundary portion between the substrate member 43 and the other member due to a difference in thermal expansion coefficient between the members. In the present embodiment, the portion that applies heat on the first opposing face 16 is formed by the substrate member 43 to which heat from the heat generating element 40 is directly transferred. Therefore, in the heat transfer path from the heat generating element 40 to the first opposing face 16, there is no boundary portion between the substrate member 43 and the other member. For this reason, the heat generated by the heat generating element 40 is transferred from the bottom surface 45 of the substrate member 43 to the first opposing face 16 (substrate facing surface 47) without any other member interposed therebetween. As a result, in comparison with a case where a portion that applies heat in the first opposing face 16 is formed by a member different from the substrate member 43, warpage or breakage due to a difference in coefficient of thermal expansion between members at the boundary portion between the members is prevented. As a result, the efficiency of transferring the heat generated by the heat generating element to the treatment target (opposing face) is prevented from being lowered, and the treatment performance of the energy treatment instrument 1 is secured.

### First Modification of First Embodiment

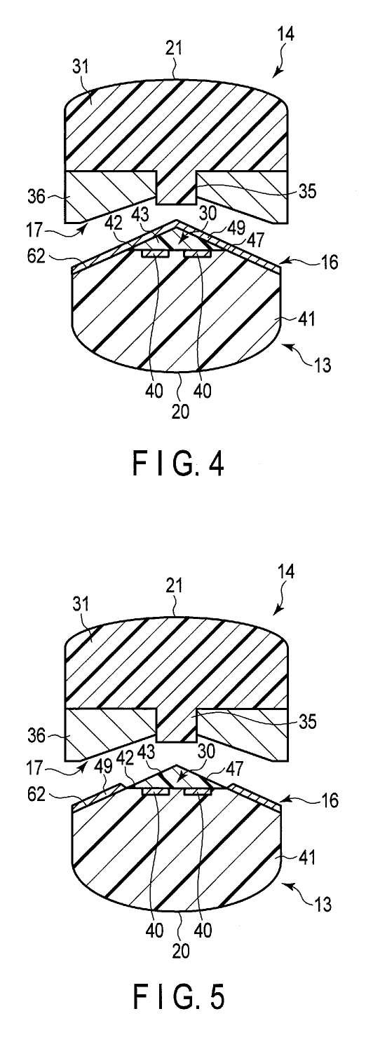

FIG. 4 is a diagram showing the first grasping piece 13 and the second grasping piece 14 in the first modification of the first embodiment. FIG. 4 shows a cross section substantially perpendicular to the longitudinal axis C. As shown in FIG. 4, the substrate facing surface 47 of the substrate member 43 may form only a part of the first opposing face 16. In FIG. 4, the insulating coating 50 is omitted.

In this modification, the support surface 42 of the base 41 is formed in a planar shape and is provided in the central portion of the base 41 in the width direction. Further, the base 41 is provided with inclined surface portions 62 provided on both outer sides of the support surface 42. The heat generating portion 30 including the substrate member 43 and the heat generating element 40 is fixed to the support surface 42 from the side of the second grasping piece 14. The support surface 42 is sandwiched between the inclined surface portions 62 from both outer ends in the width direction. The inclined surface portions 62 are formed so that the outermost ends in the width direction are closer to the back surface 20 than the ends in the center in the width direction. That is, the inclined surface portions 62 are slopes (inclined surfaces) inclined with respect to the width direction. The inclined surface portions 62 form a part of the first opposing face 16. In the present modification, the first opposing face 16 is formed by the substrate facing surface 47 of the substrate member 43 and the inclined surface portions 62 of the base 41. That is, the inclined surface portions 62 of the base 41 form a portion of the first opposing face 16 other than the portion formed by the substrate facing surface 47 of the substrate member 43.

Also in this modification, the first opposing face 16 (the substrate facing surface 47 and the inclined surface portions 62) is coated with a conductive coating (an electrical conductive coating) 49.

In this modification, the central portion in the width direction of the first opposing face 16 is formed by the substrate facing surface 47 of the substrate member 43. The side portions located on both outer sides of the central portion of the first opposing face 16 are formed by the inclined surface portions 62 of the base 41. Here, the base 41 has a lower thermal conductivity than the substrate member 43. For this reason, the heat generated by the heat generating element (heat source) 40 is intensively transferred to the central portion formed by the substrate member 43. That is, the portion where heat is intensively transferred on the first opposing face 16 is limited to the central portion formed by the substrate member 43. Since the portion where heat is intensively transferred on the first opposing face 16 is limited to the central portion, the heat is prevented from being transferred to the portion located in the side portions of the living tissue. As a result, heat invasion to an unintended part is reduced. In addition, since the portion where heat is intensively transferred on the first opposing face 16 is limited to the central portion, the residual heat on the side surfaces of the first grasping piece 13 is reduced.

### Second Modification of First Embodiment

As a second modification of the first embodiment, as shown in FIG. 5, the conductive coating 49 may be provided only on a part of the first opposing face 16. In this modification, on the first opposing face 16, the conductive coating 49 is provided in a portion formed by the inclined surface portion 62 of the base 41, but is not provided in a portion formed by the substrate facing surface 47 of a substrate member 43. Thus, the conductive coating 49 is provided only at the portion formed by the base 41 on the first opposing face 16. Therefore, in the living tissue grasped between the grasping pieces 13 and 14, a high-frequency current is applied to a portion between the inclined surface portion 62 of the base 41 and the electrode surface 37, that is, a side portion of the first opposing face 16 in the width direction.

As described above, in the present modification, heat is applied to the grasped living tissue at the central portion of the first opposing face 16, and a high-frequency current is applied to the side portion. In this way, by adjusting the portion coated with the conductive coating 49 on the first opposing face 16, the portion that applies heat to the grasped living tissue and the portion that applies a high-frequency current can be adjusted to appropriate positions.

### Third Modification of the First Embodiment

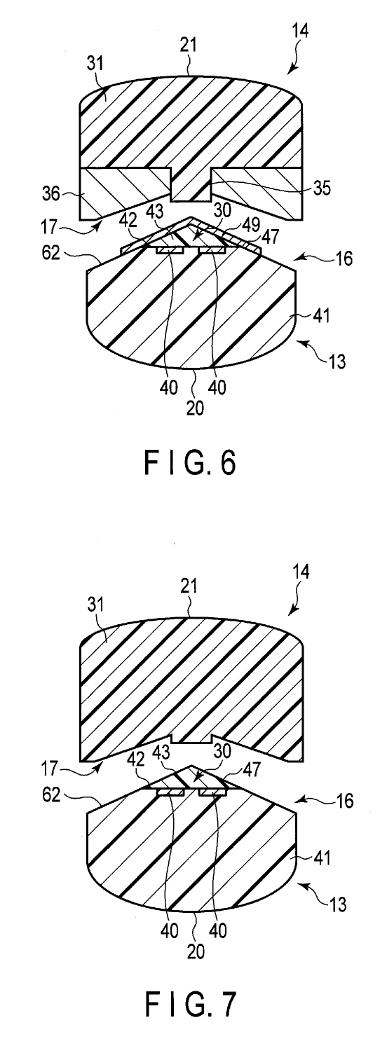

FIG. 6 is a diagram showing the first grasping piece 13 and the second grasping piece 14 in the third modification of the first embodiment. FIG. 6 shows a cross section substantially perpendicular to the longitudinal axis C. As shown in FIG. 6, the first opposing face 16 is formed by the substrate facing surface 47 of the substrate member 43 and the inclined surface portion 62 of the base 41. The substrate facing surface 47 forms a central portion of the first opposing face 16 in the width direction. In addition, the conductive coating 49 is provided only on a part of the first opposing face 16. In the present modification, the conductive coating 49 is provided continuously from the substrate facing surface 47 of the substrate member 43 to a part of the inclined surface portion 62 of the base 41 on the first opposing face 16. Thus, the conductive coating 49 is provided in a central portion of the first opposing face 16 in the width direction.

In this modification, a high-frequency current is applied to a living tissue grasped between the grasping pieces 13 and 14 at the central portion of the first opposing face 16 in the width direction. Further, the heat generated by the heat generating element 40 is intensively transferred to the central portion formed by the substrate facing surface 47. Therefore, at the central portion of the first opposing face 16, both heat and high-frequency current can be applied to the grasped living tissue.

Furthermore, in this modification, the conductive coating 49 is provided at the boundary between the substrate facing surface 47 and the inclined surface portion 62 on the first opposing face 16. Therefore, water or the like is prevented from entering a part between the substrate member 43 and the base 41 through the boundary between the substrate facing surface 47 and the inclined surface portion 62.

### Fourth Modification of the First Embodiment

Further, the configuration of the present embodiment is also applicable to an energy treatment instrument that does not apply a high-frequency current to a grasped living tissue. In this case, as shown in FIG. 7 as the fourth modification of the first embodiment, the first opposing face 16 may not be coated with the conductive coating (49). In this case, the substrate member 43 and the base 41 are exposed to the outside of the first grasping piece 13 on the first opposing face 16. Further, the second grasping piece 14 is not provided with the conductive member (36). Therefore, in this modification, the second opposing face 17 is formed only by the support member 31. In this modification, a high-frequency current is not supplied to the first opposing face 16 and the second opposing face 17. Each of the base 41 and the substrate member 43 is made of a material having an electric conductivity lower than that of the heat generating element 40.

In the above-described embodiment and the like, the protruding portion 44 may not be provided on the first opposing face 16. That is, the first opposing face 16 does not necessarily protrude toward the second grasping piece 14 side.

In the above-described embodiment and the like, the heat generating element (heat source) 40 is provided only in the first grasping piece 13, but it may be provided in both the first grasping piece 13 and the second grasping piece 14. In this case, a configuration similar to the first grasping piece 13 is also applied to the second grasping piece 14.

Additional advantages and modifications will readily occur to those skilled in the art. Therefore, the exemplary embodiments in their broader aspects are not limited to the specific details and representative embodiments shown and described herein. Accordingly, various modifications may be made without departing from the spirit or scope of the general inventive concepts.

What is claimed is:

1. An energy treatment instrument comprising:
a first grasping piece including a base plate located on an inner side of the first grasping piece, the base plate being formed of a ceramic material having electrical insulation properties;
a second grasping piece configured to open and close with respect to the first grasping piece;
a first opposing face on an outer surface of the base plate of the first grasping piece, the first opposing face being located on the inner side of the first grasping piece and facing the second grasping piece;
a second opposing face on an outer surface of the second grasping piece, the second opposing face facing the first grasping piece; and
a heat generator embedded inside the first grasping piece, the heat generator including at least a metal component, the heat generator being configured to generate heat based on a current flowing through the metal component, the base plate being configured to transfer the heat generated by the heat generator to the first opposing face.

2. The energy treatment instrument according to claim 1, wherein the base plate is formed of aluminum nitride.

3. The energy treatment instrument according to claim 1, further comprising an insulating coating disposed between the base plate and the heat generator, the insulating coating being configured to electrically insulate between the base plate and the heat generator.

4. The energy treatment instrument according to claim 1, wherein the base plate is exposed to an outside of the energy treatment instrument on the first opposing face.

5. The energy treatment instrument according to claim 1, wherein a central portion of the first opposing face in a width direction of the first grasping piece is inclined and protrudes toward the second grasping piece.

6. The energy treatment instrument according to claim 1, further comprising a conductive coating formed of a coating material disposed on at least a part of the first opposing face, the conductive coating having a water repellency and an electric conductivity, wherein:
the second grasping piece includes an electric conductive member forming at least a part of the second opposing face; and
each of the coating material and the electric conductive member functions as an electrode configured to treat a living tissue held between the first grasping piece and the second grasping piece.

7. The energy treatment instrument according to claim 5, wherein the base plate forms the central portion of the first opposing face in the width direction of the first grasping piece.

8. The energy treatment instrument according to claim 1, wherein:
the first grasping piece includes a base formed of a material having a lower thermal conductivity than the base plate and a lower electric conductivity than the heat generator; and
the heat generator is arranged between the base and the base plate.

9. The energy treatment instrument according to claim 8, wherein the base forms a portion of the first opposing face other than a portion formed by the base plate.

10. The energy treatment instrument according to claim 8, wherein the base is formed of a material that has electrical insulation properties.

11. The energy treatment instrument according to claim 8, wherein:
a central portion in the width direction of the first opposing face is formed of the base plate; and
both sides relative to the central portion on the first opposing face are formed of the base.

12. The energy treatment instrument according to claim 11, further comprising a conductive coating provided on the central portion in the width direction and on both sides relative to the central portion on the first opposing face.

13. A heat generating device for an energy treatment instrument, the heat generating device comprising:
a heat generator including at least a metal component, the heat generator being configured to generate heat based on a current flowing through the metal component of the heat generator; and
a base plate including a treatment surface located on an outer side of the base plate, the treatment surface being configured to treat a treatment target by transferring the heat generated by the heat generator to the treatment target, and the base plate including a bottom surface that is located on an opposite surface of the base plate as the treatment surface, the bottom surface being fixed to the heat generator.

14. The heat generating device according to claim 13, wherein the treatment surface of the base plate is coated with a conductive coating formed of a coating material having water repellency and electric conductivity.

15. An energy treatment instrument comprising:
a first grasping member including: (i) a first opposing face located on an outer surface of the first grasping member, (ii) a base plate located on an inner side of the first grasping member, the base plate being formed of a ceramic material having electrical insulation properties, and (iii) a base located on an outer side of the first grasping member;
a second grasping member including a second opposing face located on an outer surface of the second grasping member, the first opposing face facing the second opposing face, the second grasping member being configured to open and close with respect to the first grasping member; and
a heat generator embedded in the first grasping member on an outer side of the first grasping member and on an opposite side of the base plate as the first opposing face, the heat generator including at least a metal component and that is configured to generate heat by a current flowing through the metal component, and the base plate being configured to transfer the heat generated by the heat generator to the first opposing surface due to the base of the first grasping member having a lower thermal conductivity than a thermal conductivity of the heat generator and a thermal conductivity of the base plate.
